(19) United States
(12) Patent Application Publication
(10) Pub. No.: US 2021/0300835 A1
(43) Pub. Date: Sep. 30, 2021

(54) A Bacterial Composition Capable of Degrading Complex Carbohydrates

(71) Applicant: Monaghan Mushrooms Ireland, County Monaghan (IE)

(72) Inventors: Jude Wilson, County Monaghan (IE); Andrea Uccello, County Monaghan (IE)

(21) Appl. No.: 17/265,481

(22) PCT Filed: Aug. 8, 2019
(86) PCT No.: PCT/EP2019/071373

§ 371 Date: Feb. 2, 2021

(30) Foreign Application Priority Data
Aug. 8, 2018 (EP) 18188108.7

(51) Int. Cl. C05F 17/20; C05F 11/08; C12N 1/20

(52) U.S. Cl. CPC C05F 17/20; C12R 2001/10; C12N 1/20; C05F 11/08

(57) ABSTRACT

The present invention relates to a bacterial composition, and can be used to augment compost. According to the present invention, there is provided a bacterial composition comprising at least one strain of Bacillus licheniformis that is capable of (a) degrading complex carbohydrates, lignin, and protein; and (b) assimilating ammonia; wherein the at least one strain of Bacillus licheniformis comprises a gram positive, catalase positive and oxidase positive rod shaped strain that has a white coloured, rough surfaced colony morphology.

### FIELD OF THE INVENTION

The present invention relates to a bacterial composition, and can be used to augment compost.

### BACKGROUND OF THE INVENTION

Compost for the cultivation of Agaricus bisporus is formulated from wheat straw, poultry manure, horse manure, gypsum and ammonium sulphate. The raw materials undergo a three phase composting process to prepare a substrate that is selective for the cultivation of A. bisporus. Phase 1 involves the fermentation of the raw materials. This is characterised by the biochemical breakdown of the complex organic matter by microorganisms, which causes changes in the compost chemistry. At this stage, high temperatures (≥75° C.) are reached due to the high microbial activity. Phase 2 involves the pasteurisation of the Phase 1 compost to destroy any potential human pathogens and the removal of ammonia, which is detrimental to mushroom growth, from the compost (to less than 5 ppm). Phase 3 involves the inoculation of the Phase 2 compost with grain spawn (which contains A. bisporus spores) and the colonisation of the compost by the A. bisporus mycelium.

Mushroom compost contains a broad composition of complex carbohydrates, such as lignin, hemicellulose and cellulose, which are continually degraded throughout the cropping cycle. The degradation of these complex polymers into utilisable sugar monomers requires a variety of enzymes. These carbohydrate hydrolysis enzyme activities in compost vary throughout the production process. However, little is currently known about the particular micro-organisms that mediate these enzymatic processes.

Several authors have demonstrated that bacteria that are commonly present in mushroom compost exhibit enzymatic activities in the breakdown of complex carbon sources such as lignin, cellulose and hemicellulose. Moreover, it has been suggested that some fungi may also promote mushroom growth through the digestion of these complex carbohydrates and the subsequent release of CO₂, which has been correlated with mushroom biomass increase.

Several attempts have been made to improve the content of simple sugars that are easily utilised by Agaricus bisporus mycelium in the mushroom compost. Both fungi and bacteria have been used, with some encouraging results regarding improvements to the total composting time and final mushroom yield. Such studies were carried out at a small scale, under controlled conditions, and focused their attention only on thermophilic microorganisms. No data are available about the success of these microorganisms to augment compost at commercial scale.

### SUMMARY OF THE INVENTION

In this specification, it will be understood that the terms ‘comprise’ ‘comprises’ or ‘comprising’ may be replaced by the terms ‘consists of’, ‘consisting of’, ‘consists essentially of or consisting essentially of’.

In this specification, it will be understood that complex carbohydrates are polysaccharides, ideally containing alpha or beta type glycosidic bonds, and have longer chains compared to simple carbohydrates.

According to the present invention, there is provided a bacterial composition comprising at least one strain of Bacillus licheniformis. According to a preferred embodiment of the invention, there is also provided a bacterial composition comprising at least one strain of Bacillus licheniformis comprising the Bacillus licheniformis strain deposited at NCIMB Ltd. with accession number NCIMB 42690 and/or the Bacillus licheniformis strain deposited at NCIMB Ltd. with accession number NCIMB 42691.

Optionally, the bacterial composition is capable of degrading complex carbohydrates. Optionally the complex carbohydrates are selected from the group comprising: hemicellulose, cellulose, and combinations thereof.

Optionally, the bacterial composition is further capable of degrading lignin. Optionally, the bacterial composition is further capable of degrading protein.

Optionally, the bacterial composition is further capable of assimilating ammonia.

Optionally, the bacterial composition comprises at least one strain of Bacillus licheniformis that is capable of (a) degrading complex carbohydrates, lignin, and protein; and (b) assimilating ammonia.

Optionally, the at least one strain of Bacillus licheniformis is gram positive. Further optionally, the at least one strain of Bacillus licheniformis is catalase positive. Further optionally, the at least one strain of Bacillus licheniformis is oxidase positive.

Optionally, the at least one strain of Bacillus licheniformis is gram positive, catalase positive, and oxidase positive. Further optionally, the at least one strain of Bacillus licheniformis is gram positive and catalase positive. Further optionally, the at least one strain of Bacillus licheniformis is gram positive and oxidase positive. Optionally, the at least one strain of Bacillus licheniformis is catalase positive and oxidase positive.

Optionally, the at least one strain of Bacillus licheniformis is rod shaped. Optionally, in particular the cells of the at least one strain of Bacillus licheniformis are rod shaped. Further optionally, the cells of the at least one strain of Bacillus licheniformis have the appearance of short bacilli, with filaments.

Optionally, the at least one strain of Bacillus licheniformis comprises a gram positive, catalase positive and oxidase positive rod shaped strain.

Optionally, the at least one strain of Bacillus licheniformis comprises a gram positive, catalase positive and oxidase positive strain, the cells of which have the appearance of short bacilli, with filaments.

Optionally, the at least one strain of Bacillus licheniformis has a white coloured colony morphology. Further optionally, the at least one strain of Bacillus licheniformis has a rough surfaced colony morphology.

Optionally, the at least one strain of Bacillus licheniformis comprises a gram positive, catalase positive and oxidase positive rod shaped strain that has a white coloured, rough surfaced colony morphology. Optionally, the at least one strain of Bacillus licheniformis comprises a gram positive, catalase positive and oxidase positive rod shaped strain that has a white coloured colony morphology. Optionally, the at least one strain of Bacillus licheniformis comprises a gram positive, catalase positive and oxidase positive rod shaped strain that has a rough surfaced colony morphology.

Optionally, the at least one strain of Bacillus licheniformis has an irregular form colony morphology. Further optionally, the at least one strain of Bacillus licheniformis has a flat elevation colony morphology. Further optionally, the at least one strain of Bacillus licheniformis has a curled margin colony morphology.

Optionally, the at least one strain of Bacillus licheniformis comprises a gram positive, catalase positive and oxidase positive rod shaped strain that has an irregular form colony morphology. Further optionally, the at least one strain of Bacillus licheniformis comprises a gram positive, catalase positive and oxidase positive rod shaped strain that has a white coloured, rough surfaced, irregular form colony morphology.

Optionally, the at least one strain of Bacillus licheniformis comprises a gram positive, catalase positive and oxidase positive rod shaped strain that has a flat elevation colony morphology. Further optionally, the at least one strain of Bacillus licheniformis comprises a gram positive, catalase positive and oxidase positive rod shaped strain that has a white coloured, rough surfaced, flat elevation colony morphology.

Optionally, the at least one strain of Bacillus licheniformis comprises a gram positive, catalase positive and oxidase positive rod shaped strain that has a curled margin colony morphology. Further optionally, the at least one strain of Bacillus licheniformis comprises a gram positive, catalase positive and oxidase positive rod shaped strain that has a white coloured, rough surfaced, curled margin colony morphology.

Optionally, the at least one strain of Bacillus licheniformis has a filamentous form colony morphology. Further optionally, the at least one strain of Bacillus licheniformis has a raised elevation colony morphology. Further optionally, the at least one strain of Bacillus licheniformis has a filamentous margin colony morphology.

Optionally, the at least one strain of Bacillus licheniformis comprises a gram positive, catalase positive and oxidase positive rod shaped strain that has a filamentous form colony morphology. Further optionally, the at least one strain of Bacillus licheniformis comprises a gram positive, catalase positive and oxidase positive rod shaped strain that has a white coloured, rough surfaced, filamentous form colony morphology.

Optionally, the at least one strain of Bacillus licheniformis comprises a gram positive, catalase positive and oxidase positive rod shaped strain that has a raised elevation colony morphology. Further optionally, the at least one strain of Bacillus licheniformis comprises a gram positive, catalase positive and oxidase positive rod shaped strain that has a white coloured, rough surfaced, raised elevation colony morphology.

Optionally, the at least one strain of Bacillus licheniformis comprises a gram positive, catalase positive and oxidase positive rod shaped strain that has a filamentous margin colony morphology. Further optionally, the at least one strain of Bacillus licheniformis comprises a gram positive, catalase positive and oxidase positive rod shaped strain that has a white coloured, rough surfaced, filamentous margin colony morphology.

Optionally, the bacterial composition comprises at least one strain of Bacillus licheniformis that is capable of (a) degrading complex carbohydrates, lignin, and protein; and (b) assimilating ammonia; wherein the at least one strain of Bacillus licheniformis comprises a gram positive, catalase positive and oxidase positive rod shaped strain that has a white coloured, rough surfaced colony morphology.

Optionally, the at least one strain of Bacillus licheniformis comprises a strain selected from the group comprising:
(i) a Bacillus licheniformis strain having a white colour, rough surface, irregular form, flat elevation, and curled margin colony morphology;
(ii) a Bacillus licheniformis strain having a white colour, rough surface, filamentous form, raised elevation, and filamentous margin colony morphology; and
(iii) combinations thereof.

Optionally, the at least one strain of Bacillus licheniformis comprises a Bacillus licheniformis strain having a white colour, rough surface, irregular form, flat elevation, and curled margin colony morphology.

Optionally, the at least one strain of Bacillus licheniformis comprises a Bacillus licheniformis strain having a white colour, rough surface, filamentous form, raised elevation, and filamentous margin colony morphology.

Optionally, the bacterial composition comprises both:
(i) a Bacillus licheniformis strain having a white colour, rough surface, irregular form, flat elevation, and curled margin colony morphology; and
(ii) a Bacillus licheniformis strain having a white colour, rough surface, filamentous form, raised elevation, and filamentous margin colony morphology.

Optionally, the at least one strain of Bacillus licheniformis comprises a strain selected from the group comprising the Bacillus licheniformis strain deposited at NCIMB Ltd. with accession number NCIMB 42690, the Bacillus licheniformis strain deposited at NCIMB Ltd. with accession number NCIMB 42691, and combinations thereof.

Optionally, the at least one strain of Bacillus licheniformis comprises the Bacillus licheniformis strain deposited at NCIMB Ltd. with accession number NCIMB 42690.

Optionally, the at least one strain of Bacillus licheniformis comprises the Bacillus licheniformis strain deposited at NCIMB Ltd. with accession number NCIMB 42691.

Optionally, the bacterial composition comprises or consists of or consists essentially of both the Bacillus licheniformis strain deposited at NCIMB Ltd. with accession number NCIMB 42690, and the Bacillus licheniformis strain deposited at NCIMB Ltd. with accession number NCIMB 42691.

According to the present invention, there is provided a kit of parts for use in preparing augmented compost and/or farming, the kit of parts comprising the bacterial composition.

Optionally, there is provided a kit of parts for use in preparing augmented compost and/or farming, the kit of parts comprising the bacterial composition comprising at least one strain of Bacillus licheniformis.

Optionally, there is provided a kit of parts for use in preparing augmented compost and/or farming, the kit of parts comprising the bacterial composition comprising at least one strain of Bacillus licheniformis that is capable of (a) degrading complex carbohydrates, lignin, and protein; and (b) assimilating ammonia; wherein the at least one strain of Bacillus licheniformis comprises a gram positive, catalase positive and oxidase positive rod shaped strain that has a white coloured, rough surfaced colony morphology.

Optionally, there is provided a kit of parts for use in preparing augmented compost and/or farming, the kit of parts comprising the bacterial composition comprising a strain selected from the group comprising:
a. a Bacillus licheniformis strain having a white colour, rough surface, irregular form, flat elevation, and curled margin colony morphology;
b. a Bacillus licheniformis strain having a white colour, rough surface, filamentous form, raised elevation, and filamentous margin colony morphology; and
c. combinations thereof.

Optionally, there is provided a kit of parts for use in preparing augmented compost and/or farming, the kit of parts comprising the bacterial composition comprising a Bacillus licheniformis strain having a white colour, rough surface, irregular form, flat elevation, and curled margin colony morphology.

Optionally, there is provided a kit of parts for use in preparing augmented compost and/or farming, the kit of parts comprising the bacterial composition comprising a Bacillus licheniformis strain having a white colour, rough surface, filamentous form, raised elevation, and filamentous margin colony morphology.

Optionally, there is provided a kit of parts for use in preparing augmented compost and/or farming, the kit of parts comprising the bacterial composition comprising both:
a. a Bacillus licheniformis strain having a white colour, rough surface, irregular form, flat elevation, and curled margin colony morphology; and
b. a Bacillus licheniformis strain having a white colour, rough surface, filamentous form, raised elevation, and filamentous margin colony morphology.

Optionally, there is provided a kit of parts for use in preparing augmented compost and/or farming, the kit of parts comprising the bacterial composition comprising a strain selected from the group comprising: the Bacillus licheniformis strain deposited at NCIMB Ltd. with accession number NCIMB 42690, the Bacillus licheniformis strain deposited at NCIMB Ltd. with accession number NCIMB 42691, and combinations thereof.

Optionally, there is provided a kit of parts for use in preparing augmented compost and/or farming, the kit of parts comprising the bacterial composition comprising the Bacillus licheniformis strain deposited at NCIMB Ltd. with accession number NCIMB 42690.

Optionally, there is provided a kit of parts for use in preparing augmented compost and/or farming, the kit of parts comprising the bacterial composition comprising the Bacillus licheniformis strain deposited at NCIMB Ltd. with accession number NCIMB 42691.

Optionally, there is provided a kit of parts for use in preparing augmented compost and/or farming, the kit of parts comprising the bacterial composition comprising the Bacillus licheniformis strain deposited at NCIMB Ltd. with accession number NCIMB 42690, and the Bacillus licheniformis strain deposited at NCIMB Ltd. with accession number NCIMB 42691.

Optionally, the kit of parts further comprises instructions for use thereof.

According to the present invention, there is provided a method for preparing augmented compost, the method comprising the steps of:
(i) providing the bacterial composition;
(ii) providing compost;
(iii) inoculating the compost with the bacterial composition, optionally under temperature-controlled conditions, optionally under aerobic conditions.

Optionally, the method of preparing augmented compost comprises the steps of:
(i) providing the bacterial composition comprising at least one strain of Bacillus licheniformis;
(ii) providing compost;
(iii) inoculating the compost with the bacterial composition, optionally under temperature-controlled conditions, optionally under aerobic conditions.

Optionally, the method of preparing augmented compost comprises the steps of:
(i) providing the bacterial composition comprising at least one strain of Bacillus licheniformis that is capable of (a) degrading complex carbohydrates, lignin, and protein; and (b) assimilating ammonia; wherein the at least one strain of Bacillus licheniformis comprises a gram positive, catalase positive and oxidase positive rod shaped strain that has a white coloured, rough surfaced colony morphology;
(ii) providing compost;
(iii) inoculating the compost with the bacterial composition, optionally under temperature-controlled conditions, optionally under aerobic conditions.

Optionally, the method of preparing augmented compost comprises the steps of:
(i) providing the bacterial composition comprising a strain selected from the group comprising:
   d. a Bacillus licheniformis strain having a white colour, rough surface, irregular form, flat elevation, and curled margin colony morphology;
   e. a Bacillus licheniformis strain having a white colour, rough surface, filamentous form, raised elevation, and filamentous margin colony morphology; and
   f. combinations thereof;
(ii) providing compost;
(iii) inoculating the compost with the bacterial composition, optionally under temperature-controlled conditions, optionally under aerobic conditions.

Optionally, the method of preparing augmented compost comprises the steps of:
(i) providing the bacterial composition comprising a Bacillus licheniformis strain having a white colour, rough surface, irregular form, flat elevation, and curled margin colony morphology;
(ii) providing compost;
(iii) inoculating the compost with the bacterial composition, optionally under temperature-controlled conditions, optionally under aerobic conditions.

Optionally, the method of preparing augmented compost comprises the steps of:
(i) providing the bacterial composition comprising a Bacillus licheniformis strain having a white colour, rough surface, filamentous form, raised elevation, and filamentous margin colony morphology; and
(ii) providing compost;
   inoculating the compost with the bacterial composition, optionally under temperature-controlled conditions, optionally under aerobic conditions.

Optionally, the method of preparing augmented compost comprises the steps of:
(i) providing the bacterial composition comprising both:
   c. a Bacillus licheniformis strain having a white colour, rough surface, irregular form, flat elevation, and curled margin colony morphology; and
   d. a Bacillus licheniformis strain having a white colour, rough surface, filamentous form, raised elevation, and filamentous margin colony morphology;
(ii) providing compost;
(iii) inoculating the compost with the bacterial composition, optionally under temperature-controlled conditions, optionally under aerobic conditions.

Optionally, the method of preparing augmented compost comprises the steps of:
(i) providing the bacterial composition comprising a strain selected from the group comprising: the Bacillus licheniformis strain deposited at NCIMB Ltd. with accession number NCIMB 42690, the Bacillus licheniformis strain deposited at NCIMB Ltd. with accession number NCIMB 42691, and combinations thereof;
(ii) providing compost;
(iii) inoculating the compost with the bacterial composition, optionally under temperature-controlled conditions, optionally under aerobic conditions.

Optionally, the method of preparing augmented compost comprises the steps of:
(i) providing the bacterial composition comprising the Bacillus licheniformis strain deposited at NCIMB Ltd. with accession number NCIMB 42690;
(ii) providing compost;
(iii) inoculating the compost with the bacterial composition, optionally under temperature-controlled conditions, optionally under aerobic conditions.

Optionally, the method of preparing augmented compost comprises the steps of:
(i) providing the bacterial composition comprising the Bacillus licheniformis strain deposited at NCIMB Ltd. with accession number NCIMB 42691;
(ii) providing compost;
(iii) inoculating the compost with the bacterial composition, optionally under temperature-controlled conditions, optionally under aerobic conditions.

Optionally, the method of preparing augmented compost comprises the steps of:
(i) providing the bacterial composition comprising both the Bacillus licheniformis strain deposited at NCIMB Ltd. with accession number NCIMB 42690, and the Bacillus licheniformis strain deposited at NCIMB Ltd. with accession number NCIMB 42691;
(ii) providing compost;
(iii) inoculating the compost with the bacterial composition, optionally under temperature-controlled conditions, optionally under aerobic conditions.

Optionally, the method for preparing augmented compost is carried out under temperature-controlled conditions.

Optionally, the method for preparing augmented compost is carried out under aerobic conditions.

Optionally, the method for preparing augmented compost is carried out under temperature-controlled and aerobic conditions.

Optionally, the step of inoculating the compost with the bacterial composition is carried out under temperature-controlled conditions.

Optionally, the step of inoculating the compost with the bacterial composition is carried out under aerobic conditions.

Optionally, the step of inoculating the compost with the bacterial composition is carried out under temperature-controlled and aerobic conditions.

According to the present invention, there is provided augmented compost prepared according to the method of preparing augmented compost.

Optionally, there is provided augmented compost prepared according to a method for preparing augmented compost, the method comprising the steps of:
(i) providing the bacterial composition;
(ii) providing compost;
(iii) inoculating the compost with the bacterial composition, optionally under temperature-controlled conditions, optionally under aerobic conditions.

Optionally, there is provided augmented compost prepared according to a method for preparing augmented compost, the method comprising the steps of:
(i) providing the bacterial composition comprising at least one strain of Bacillus licheniformis;
(ii) providing compost;
   inoculating the compost with the bacterial composition, optionally under temperature-controlled conditions, optionally under aerobic conditions.

Optionally, there is provided augmented compost prepared according to a method for preparing augmented compost, the method comprising the steps of:
(i) providing the bacterial composition comprising at least one strain of Bacillus licheniformis that is capable of (a) degrading complex carbohydrates, lignin, and protein; and (b) assimilating ammonia; wherein the at least one strain of Bacillus licheniformis comprises a gram positive, catalase positive and oxidase positive rod shaped strain that has a white coloured, rough surfaced colony morphology;
(ii) providing compost;
   inoculating the compost with the bacterial composition, optionally under temperature-controlled conditions, optionally under aerobic conditions.

Optionally, there is provided augmented compost prepared according to a method for preparing augmented compost, the method comprising the steps of:
(i) providing the bacterial composition comprising a strain selected from the group comprising:
   a. a Bacillus licheniformis strain having a white colour, rough surface, irregular form, flat elevation, and curled margin colony morphology;
   b. a Bacillus licheniformis strain having a white colour, rough surface, filamentous form, raised elevation, and filamentous margin colony morphology; and
   c. combinations thereof;
(ii) providing compost;
   inoculating the compost with the bacterial composition, optionally under temperature-controlled conditions, optionally under aerobic conditions.

Optionally, there is provided augmented compost prepared according to a method for preparing augmented compost, the method comprising the steps of:
(i) providing the bacterial composition comprising a Bacillus licheniformis strain having a white colour, rough surface, irregular form, flat elevation, and curled margin colony morphology;
(ii) providing compost;
   inoculating the compost with the bacterial composition, optionally under temperature-controlled conditions, optionally under aerobic conditions.

Optionally, there is provided augmented compost prepared according to a method for preparing augmented compost, the method comprising the steps of:
(i) providing the bacterial composition comprising a Bacillus licheniformis strain having a white colour, rough surface, filamentous form, raised elevation, and filamentous margin colony morphology;
(ii) providing compost;
   inoculating the compost with the bacterial composition, optionally under temperature-controlled conditions, optionally under aerobic conditions.

Optionally, there is provided augmented compost prepared according to a method for preparing augmented compost, the method comprising the steps of:
(i) providing the bacterial composition comprising both:
   a. a Bacillus licheniformis strain having a white colour, rough surface, irregular form, flat elevation, and curled margin colony morphology; and
   b. a Bacillus licheniformis strain having a white colour, rough surface, filamentous form, raised elevation, and filamentous margin colony morphology;
(ii) providing compost;
(iii) inoculating the compost with the bacterial composition, optionally under temperature-controlled conditions, optionally under aerobic conditions.

Optionally, there is provided augmented compost prepared according to a method for preparing augmented compost, the method comprising the steps of:
(i) providing the bacterial composition comprising a strain selected from the group comprising: the Bacillus licheniformis strain deposited at NCIMB Ltd. with accession number NCIMB 42690, the Bacillus licheniformis strain deposited at NCIMB Ltd. with accession number NCIMB 42691, and combinations thereof;
(ii) providing compost;
(iii) inoculating the compost with the bacterial composition, optionally under temperature-controlled conditions, optionally under aerobic conditions.

Optionally, there is provided augmented compost prepared according to a method for preparing augmented compost, the method comprising the steps of:
(i) providing the bacterial composition comprising the Bacillus licheniformis strain deposited at NCIMB Ltd. with accession number NCIMB 42690;
(ii) providing compost;
(iii) inoculating the compost with the bacterial composition, optionally under temperature-controlled conditions, optionally under aerobic conditions.

Optionally, there is provided augmented compost prepared according to a method for preparing augmented compost, the method comprising the steps of:
(i) providing the bacterial composition comprising the Bacillus licheniformis strain deposited at NCIMB Ltd. with accession number NCIMB 42691;
(ii) providing compost;
(iii) inoculating the compost with the bacterial composition, optionally under temperature-controlled conditions, optionally under aerobic conditions.

Optionally, there is provided augmented compost prepared according to a method for preparing augmented compost, the method comprising the steps of:
(i) providing the bacterial composition comprising both the Bacillus licheniformis strain deposited at NCIMB Ltd. with accession number NCIMB 42690, and the Bacillus licheniformis strain deposited at NCIMB Ltd. with accession number NCIMB 42691;
(ii) providing compost;
(iii) inoculating the compost with the bacterial composition, optionally under temperature-controlled conditions, optionally under aerobic conditions.

Optionally, the augmented compost is prepared under temperature-controlled conditions.

Optionally, the augmented compost is prepared under aerobic conditions.

Optionally, the augmented compost is prepared under temperature-controlled and aerobic conditions.

Optionally, the augmented compost is prepared according to a method for preparing augmented compost comprising the step of inoculating the compost with the bacterial composition under temperature-controlled conditions.

Optionally, the augmented compost is prepared according to a method for preparing augmented compost comprising the step of inoculating the compost with the bacterial composition is carried out under aerobic conditions.

Optionally, the augmented compost is prepared according to a method for preparing augmented compost comprising the step of inoculating the compost with the bacterial composition is carried out under temperature-controlled and aerobic conditions.

According to the present invention, there is provided augmented compost comprising the bacterial composition.

Optionally, there is provided augmented compost comprising the bacterial composition comprising at least one strain of Bacillus licheniformis.

Optionally, there is provided augmented compost comprising the bacterial composition comprising at least one strain of Bacillus licheniformis that is capable of (a) degrading complex carbohydrates, lignin, and protein; and (b) assimilating ammonia; wherein the at least one strain of Bacillus licheniformis comprises a gram positive, catalase positive and oxidase positive rod shaped strain that has a white coloured, rough surfaced colony morphology.

Optionally, there is provided augmented compost comprising the bacterial composition comprising a strain selected from the group comprising:
a. a Bacillus licheniformis strain having a white colour, rough surface, irregular form, flat elevation, and curled margin colony morphology;
b. a Bacillus licheniformis strain having a white colour, rough surface, filamentous form, raised elevation, and filamentous margin colony morphology; and
c. combinations thereof.

Optionally, there is provided augmented compost comprising the bacterial composition comprising a Bacillus licheniformis strain having a white colour, rough surface, irregular form, flat elevation, and curled margin colony morphology;

Optionally, there is provided augmented compost comprising the bacterial composition comprising a Bacillus licheniformis strain having a white colour, rough surface, filamentous form, raised elevation, and filamentous margin colony morphology.

Optionally, there is provided augmented compost comprising the bacterial composition comprising both:
a. a Bacillus licheniformis strain having a white colour, rough surface, irregular form, flat elevation, and curled margin colony morphology; and
b. a Bacillus licheniformis strain having a white colour, rough surface, filamentous form, raised elevation, and filamentous margin colony morphology.

Optionally, there is provided augmented compost comprising the bacterial composition comprising a strain selected from the group comprising: the Bacillus licheniformis strain deposited at NCIMB Ltd. with accession number NCIMB 42690, the Bacillus licheniformis strain deposited at NCIMB Ltd. with accession number NCIMB 42691, and combinations thereof.

Optionally, there is provided augmented compost comprising the bacterial composition comprising a strain selected from the group comprising the Bacillus licheniformis strain deposited at NCIMB Ltd. with accession number NCIMB 42690.

Optionally, there is provided augmented compost comprising the bacterial composition comprising the Bacillus licheniformis strain deposited at NCIMB Ltd. with accession number NCIMB 42691.

Optionally, there is provided augmented compost comprising the bacterial composition comprising both the Bacillus licheniformis strain deposited at NCIMB Ltd. with accession number NCIMB 42690, and the Bacillus licheniformis strain deposited at NCIMB Ltd. with accession number NCIMB 42691.

According to the present invention, there is provided a use of augmented compost comprising the bacterial composition as a nutrient source for farming, the use comprising the steps of:
(i) providing the augmented compost;
(ii) providing at least one fungus, mycelium, spawn, spore, plant, cutting, turf, and/or seed;
(iii) cultivating the at least one fungus, mycelium, spawn, spore, plant, cutting, turf, and/or seed in an environment comprising the augmented compost.

Optionally, there is provided a use of augmented compost comprising the bacterial composition as a nutrient source for fungus farming, the use comprising the steps of:
(i) providing the augmented compost;
(ii) providing at least one fungus, mycelium, spawn, and/or spore;
(iii) cultivating the at least one fungus, mycelium, spawn, and/or spore, in an environment comprising the augmented compost.

Optionally, there is provided a use of augmented compost comprising the bacterial composition as a nutrient source for mushroom farming, the use comprising the steps of:
(i) providing the augmented compost;
(ii) providing at least one mushroom, mycelium, spawn, and/or spore;
(iii) cultivating the at least one mushroom, mycelium, spawn, and/or spore, in an environment comprising the augmented compost.

Optionally, there is provided a use of augmented compost comprising the bacterial composition as a nutrient source for Agaricus bisporus farming, the use comprising the steps of:
(i) providing the augmented compost;
(ii) providing at least one Agaricus bisporus mushroom, mycelium, spawn, and/or spore;
(iii) cultivating the at least one mushroom, mycelium, spawn, and/or spore, in an environment comprising the augmented compost.

According to the present invention, there is provided a use of augmented compost as a nutrient source for farming, the use comprising the steps of:
(iv) providing the augmented compost;
(v) providing at least one fungus, mycelium, spawn, spore, plant, cutting, turf, and/or seed;
(vi) cultivating the at least one fungus, mycelium, spawn, spore, plant, cutting, turf, and/or seed in an environment comprising the augmented compost.

Optionally, there is provided a use of augmented compost as a nutrient source for fungus farming, the use comprising the steps of:
(iv) providing the augmented compost;
(v) providing at least one fungus, mycelium, spawn, and/or spore;
(vi) cultivating the at least one fungus, mycelium, spawn, and/or spore, in an environment comprising the augmented compost.

Optionally, there is provided a use of augmented compost as a nutrient source for mushroom farming, the use comprising the steps of:
(iv) providing the augmented compost;
(v) providing at least one mushroom, mycelium, spawn, and/or spore;
(vi) cultivating the at least one mushroom, mycelium, spawn, and/or spore, in an environment comprising the augmented compost.

Optionally, there is provided a use of augmented compost as a nutrient source for Agaricus bisporus farming, the use comprising the steps of:
(iv) providing the augmented compost;
(v) providing at least one Agaricus bisporus mushroom, mycelium, spawn, and/or spore;
(vi) cultivating the at least one mushroom, mycelium, spawn, and/or spore, in an environment comprising the augmented compost.

For the avoidance of doubt, MM76 refers to the strain deposited under accession number NCIMB 42690. Likewise, MM172 refers to the strain deposited under accession number NCIMB 42691.

### BRIEF DESCRIPTION OF THE DRAWINGS

FIG. 1A and FIG. 1B each shows an image of the colonies formed by MM76 on TSA media.

FIG. 2A and FIG. 2B each shows an image of the colonies formed by MM172 on TSA media.

FIG. 3 shows a chart representing typical glucanase activity in control and augmented compost at day 2 and day 7 of composting at bench scale.

FIG. 4 shows charts representing the two typical scenarios observed among the different trials performed. The simple sugar production in augmented compared to control compost prepared in the bench composting system showed that: a) the levels of glucose increased significantly, but the levels of trehalose (a biosynthetic product of glucose) did not; b) the levels of glucose did not rise detectably, but the levels of trehalose were increased. This suggests that in scenario a) the compost was sampled slightly earlier in the degradation of cellulose than in scenario b).

FIG. 5 shows a chart representing laccase activity of both control and augmented compost during colonisation by Agaricus bisporus mycelium at bench scale. Results presented are an average of triplicates from three trials.

FIG. 6 shows charts representing laccase activities in control and augmented composts during colonisation by Agaricus bisporus mycelium throughout (A) Phase 3 composting and (B) during case run. Results presented are an average of triplicates from two commercial scale trials.

FIG. 7 shows a chart representing the percentage increase in mushrooms yield from the augmented compost compared to control, indicating that the highest increases in yield were predominantly from flushes 2 and 3.

FIG. 8 shows a chart representing mushroom piece weight according the cup diameter.

### DETAILED DESCRIPTION OF THE INVENTION

The inventors have isolated 389 bacterial and 210 fungal strains from compost at different stages of production and from different compost yards in Ireland, based in Co. Monaghan and Co. Kildare, respectively. All of the bacterial strains were identified and assessed for their ability to breakdown complex carbohydrates that are associated with mushroom compost (cellulose, hemicellulose and lignin). Several that scored highly were selected for further assessment of their effect in the bioaugmentation of compost, in terms of improving compost productivity.

Small microcosm and larger semi-commercial scale trials were used to confirm the beneficial effects of these on compost quality. The compost from the semi-commercial scale trials was used to grow mushrooms in a trial mushroom farm, confirming that the augmented compost consistently yielded 4% higher than un-augmented compost. Additionally, the mushrooms cultivated from augmented compost had a higher piece weight (3-5%) than those from control compost. The results of these trials were consistent and were observed in compost produced during different seasons and in different locations. It was concluded that the augmentation of compost with the MM76/MM172 microbial composition led to an increase in soluble sugars, promoting the colonisation of compost by Agaricus bisporus. This resulted in a higher yield and quality of mushrooms.

Compost for the cultivation of Agaricus bisporus is composed of complex carbohydrates such as cellulose, hemicellulose and lignin. These are gradually broken down into simple sugars during the composting process, through the activity of the microbial community within the compost. These complex carbohydrates are further broken down during the mushroom cultivation process.

One mushroom cropping cycle involves the production of three mushrooms flushes. However, the yield from each mushroom flush decreases significantly. There have been many hypotheses put forward to explain this successive reduction in mushroom yield, including a decrease in the microbial breakdown of complex carbohydrates.

In order to address this hypothesis, the inventors have developed a composition comprising at least one of two Bacillus licheniformis strains (strain MM76 (deposited under accession number NCIMB 42690) and/or strain MM172 (deposited under accession number: NCIMB 42691)); each strain was originally isolated from mushroom compost. It was demonstrated that the augmentation of compost with this microbial composition increased the cellulase activity, leading to the increased production of glucose and/or trehalose. The augmented compost, with the higher levels of soluble sugars, was better colonised with Agaricus bisporus mycelium and this consistently led to a higher yield of heavier mushrooms.

The composition could comprise strain MM76 alone, or strain MM172 alone, or both strains MM76 and MM172. Any such composition, comprising at least one of strain MM76 and/or strain MM172, is able to degrade complex carbohydrates, and/or to degrade protein, and/or to assimilate ammonia. Addition of such a composition comprising at least one of strain MM76 and/or strain MM172 to compost led to increased production of soluble sugars such as glucose and/or trehalose from complex carbohydrates within the compost. Addition of such a composition comprising at least one of strain MM76 and/or strain MM172 to compost further led to better colonisation of the augmented compost with Agaricus bisporus mycelium. Addition of such a composition comprising at least one of strain MM76 and/or strain MM172 to compost further led to a higher yield of heavier mushrooms.

A composition comprising just one of strain MM76 or strain MM172 is effective, while a composition comprising both strain MM76 and strain MM172 is also effective. The two strains MM76 and MM172 do not inhibit each other. In particular, neither strain MM76 nor strain MM172 significantly inhibited the growth of the other strain. On the contrary, in a composition comprising both strain MM76 and strain MM172, the strains have a synergistic effect. We postulate that the synergistic effect is related to the different optimal growing temperature of the two strains. The optimal temperature range for the growth of the strain MM76 only partially overlaps that for the strain MM172. The combination of the optimal temperature ranges for the growth of both isolates covers the entire temperature range of the industrial process to produce the augmented compost.

While the present invention has application in mushroom farming, in particular farming of Agaricus bisporus, is clear that a composition comprising at least one of strain MM76 and/or strain MM172 could be usefully added to compost, soil, fertilizer or another fungus or plant farming medium or growth medium; to improve the farming of any of a wide variety of fungi and/or plants, including for example edible mushrooms, button mushrooms (Agaricus bisporus), Portobello mushrooms (Agaricus bisporus), straw mushrooms (Volvariella volvacea), oyster mushrooms (Pleurotus ostreatus), shiitakes (Lentinula edodes), enokitake (Flammulina spp.), fruit, bananas, apples, pears, oranges, melons, strawberries, nuts, vegetables, potato, carrot, legume pulses, cassava, groundnuts, beans, peas, green vegetables, broccoli, salad vegetables, celery, lettuce, sweet potatoes, grasses, rice, wheat, sugarcane, maize, corn, soybean, sunflower rapeseed, mustard. Furthermore, it is clear that a composition comprising at least one of strain MM76 and/or strain MM172 could be used to augment, or in the preparation of, compost soil, fertilizer or another fungus or plant farming medium for sale. Furthermore, it is clear that a composition comprising at least one of strain MM76 and/or strain MM172 could be used in Mycoremediation. Furthermore, it is clear that a composition comprising at least one of strain MM76 and/or strain MM172 could be used in food/ingredient preparation and/or in food technology.

One of these two Bacillus licheniformis strains, referred to as MM76, was determined to be a gram positive, catalase positive and oxidase positive rod shaped bacillus. Sequencing of the 16S ribosomal RNA (SEQ ID No. 3) indicated that MM76 was a Bacillus licheniformis strain. On Nov. 16, 2016, MM76 was deposited under the Budapest Treaty in NCIMB and was confirmed as a Bacillus licheniformis strain (Accession number: NCIMB 42690).

The other of these two Bacillus licheniformis strains, referred to as MM172 was determined to be a gram positive, catalase positive and oxidase positive rod shaped bacillus. Sequencing of the 16S ribosomal RNA (SEQ ID No. 4) indicated that MM172 was a Bacillus licheniformis strain. On Nov. 16, 2016, MM172 was deposited under the Budapest Treaty in NCIMB and was confirmed as a Bacillus licheniformis strain (Accession number: NCIMB 42691).

### EXAMPLES

Isolation of Bacteria from Compost

Isolation of bacteria from compost was as follows: 10 g of Phase 1 mushroom compost was added to 90 ml of Maximum Recovery Diluent (MRD) and agitated for 30 mins at 150 rpm. Serial dilutions were prepared and spread onto different media agar plates. These were incubated at 25° C., 37° C. and 55° C. for 24-48 hrs and any colonies that appeared on the plates were sub-cultured. Pure colonies were stored as 50% glycerol stocks at −80° C. for further analysis.

Catalase and oxidase activities were assayed using hydrogen peroxide breakdown and tetramethyl- or dimethyl p-phenlyenediamine. Gram characterisation was conducted using a commercially available Gram staining kit.

Purification and Amplification of DNA

Total genomic DNA was extracted from bacterial isolates using a commercial genomic DNA extraction kit (such as the Invitrogen PureLink® Genomic DNA kit) according to the manufacturer's instructions. PCR amplification of the 16S rRNA gene was conducted for sequence identification of bacterial isolates. The DNA fragments were amplified using the primers 27f (5′-AGAGTTTGGATCMTGGCTCAG-3′) (SEQ ID NO: 1) and 1492r (5′-CGGTTACCTTGTTACGACTT-3′) (SEQ ID NO: 2) (Jiang et al., 2006). The PCR was performed using a commercial high-fidelity PCR kit (such as the Q5® Hot Start High Fidelity master mix; New England Biolabs® Inc.), in a final volume of 25 μl per reaction. The PCR condition used were the following: initial denaturation at 98° C. for 60 seconds; 35 cycles of denaturation (98° C., 10 seconds), annealing (55° C., 20 seconds) and DNA extension (72° C., 35 seconds); final DNA extension at 72° C. for 2 minutes. The amplicons obtained were sequenced both in forward and reverse. This resulted in a ˜1.5 Kbp long consensus sequence being obtained for each isolate. The sequences were queried using an online BLAST® nucleotide database (for example on the website of the National Center for Biotechnology Information of the US National Library of Medicine available at https://blast.ncbi.nlm.nih.gov/Blast.cgi) and identifications were considered reliable for results matching >98% of identity.

In Vitro Assessment of the Ability of Bacterial Strains to Degrade Complex Carbohydrates

The 389 bacterial isolates were tested for their ability to degrade four different carbon sources: hemicellulose (growth medium: 5 g/L xylan agar+1 mg/ml congo red stain after bacterial growth); proteins (growth medium: 15 g/L skim milk agar); cellulose (growth medium: 25 g/L carboxymethil-cellulose agar+1 mg/ml congo red stain after bacterial growth); lignin (growth medium: Luria-Bertani amended with congo red/toluidine blue agars). The isolates were also tested for their ability to assimilate ammonia (growth medium: glucose, NH₄Cl, ferric citrate, NaCl, MgSO₄, MnSO₄, K₂HPO₄, KH₂PO₄ agar).

Cultures were incubated at the optimal temperatures (25° C. for the low mesophiles; 37° C. for the mesophiles; 55° C. for the thermophiles) for 48 hours. For lignin and ammonia assays, colony growth was used as a parameter to estimate the lignin/ammonia utilisation. For cellulose and hemicellulose assays, the presence and the dimension of halos around the colonies was observed after washing the medium with the congo red solution, followed by a medium wash with 5M NaCl solution. For protein assay, the presence and the dimension of a halo around the bacterial colony was used to estimate the protein assimilation. The higher the colony/halo dimension, the higher the score assigned, ranging from 0 (no growth/halo) to +++(large colony/halo). The score assignment was qualitative and subjective. The total score was used to develop a numerical rating to easily identify those microbes with the greatest abilities to degrade the complex carbohydrates (0-18). The arbitrary threshold chosen to identify the potential candidates for inclusion in a microbial composition was 15.

Preparation of the Bacterial Strains for Use in Small and Large Scale Composting Trials.

Bacterial cultures were prepared using commercially available bench-scale bioreactor/fermenters system (such as Eppendorf BioFlo 120 (10 L) fermenters). Strain MM76 was fermented in Tryptic Soy Broth (TSB) at 55° C. at 25% air saturation until the end of exponential phase (˜6.5 hrs), resulting in 3×10⁹ viable cells/ml. Strain MM172 was fermented in Tryptic Soy Broth (TSB) at 37° C. at 25% air saturation until the end of exponential phase (˜6.5 hrs), resulting in 1.5×10⁹ viable cells/ml.

Small Scale Assessment of the Ability of the Bacterial Strains to Degrade Complex Carbohydrates During Mushroom Composting

The small scale assessment of the ability of the MM76/MM172 composition to increase the productivity of compost was conducted using a bench composting system (BCS) that can be used to produce mushroom compost. This system was set up following the specifications described in Noble et al. (1997), who performed the entire mushroom composting process in 2 L flasks. Those specifications were adapted to the upscaled system and adjusted until optimal results were reached. These included an initial warming up stage, to bring the temperature up to 60° C. for 12-18 hours, followed by a pasteurisation stage at temperatures between 44-48° C. and a final conditioning stage at 25° C. for 24 hours (Colak, 2004). 2.6 kg of the starter mushroom compost was filled into a 10 L culture vessel and then placed into a temperature-controlled stainless steel water bath. Prior to filling, a stainless steel platform was placed in the bottom of each flask to allow air to be driven up through the compost. The stainless steel platforms were perforated with 2 mm holes to allow the air to pass through them. Rubber tubing, which is connected to an air-flow meter, was inserted through a central hole in the platform. The temperature in the waterbath was manually changed to reach the temperature profile needed for the process. The air flow meter was used to ensure a constant air flow through the compost, to provide aerobic conditions. Vessel lids opening was managed to obtain the optimal moisture loss. A dissolved oxygen (DO)/temperature probe was inserted into the centre of the compost inside the vessel. The dissolved oxygen and temperature of the compost were measured and logged on a laptop using dedicated software. The dissolved oxygen profile was used to estimate if the proper aerobic fermentation occurred in the early composting stage, before the pasteurisation started. The composting conditions used mimicked those of the current industrial standards (Colak, 2004).

The bacterial cells, for example cells of strain MM76 and/or strain MM172, were re-suspended in a 0.9% NaCl sterile solution and applied to the compost using a sterile spray bottle. The compost was thoroughly mixed prior to filling the vessels, to ensure an even distribution of the microbial inoculum. Non-inoculated compost was sprayed with sterile 0.9% NaCl sterile solution, as a negative control.

Compost from inoculated and control vessels was sampled at day 2 and day 7 of the bench scale process for analysis of glucanase and xylanase activity. The assays were conducted by the spectrophotometric estimation of simple sugars (glucose, xylan) from the crude compost extract. This was determined by comparison with omologues sugars released by the digestion of commercial oat β-glucan and beechwood xylan at known concentrations, according to the methodology described in King et al., 2009. At the end of the process, the resultant composts were analysed for the water soluble sugar content using a high-performance anion exchange chromatography with pulsed amperometric detection (HPAEC-PAD) methodology. The high performance anion-exchange chromatography was performed on a commercially available high-performance anion exchange chromatography (HPAEC) system (such as Dionex® ICS 5000+ system (Thermo Scientific®)) equipped with a suitable analytical column (such as CarboPac® PA-1 column) in combination with a suitable guard column (such as CarboPac® guard column) and pulsed amperometric detection (PAD). The elution of sugars was performed at a flow rate of 0.75 mL min-1, combining two mobile phases: (A) 0.2 M NaOH, (B) H₂O. Water soluble sugars were extracted from freeze-dried, grinded compost samples in 50% methanol at 70° C. for 15 minutes. After centrifugation at 14000 rpm for 15 minutes, the supernatants were mixed with double-distilled water (ddH₂O). A further 1:2 H₂O dilution step was carried out, before 400 μL of the sample was filtered through a 0.2 μm nylon syringe filter and injected on to the HPAEC system.

Small Scale Assessment of the Ability of the Augmented Mushroom Compost to be Colonised by Agaricus bisporus Mycelium.

To confirm if the compost produced by the BCS could be colonised by Agaricus bisporus, 4 spawn grains of a known Agaricus bisporus strain (such as Sylvan® A15 strain spawn grains) were added to each 150 mm plate containing 50 g of the control or augmented compost. The plates were left at room temperature for 21 days to allow the mycelium to colonise the compost. Laccase enzyme activity analysis was performed comparing the reduction of ABTS (2,2′-Azino-bis(3-ethylbenzothiazoline-6-sulfonic acid) by the compost crude extract with that obtained from a known concentration of a commercial laccase enzyme, followed by spectrophotometry analysis, according to the methodology described in Johannes and Majcherczyk, 2000.

Large Scale Assessment of the Ability of the Bacterial Strains to Degrade Complex Carbohydrates During Mushroom Composting.

The freeze dried inoculum was re-suspended in 60 L of tap water and left to re-hydrate for 60 minutes, with occasional stirring. 140 tonnes of compost were inoculated, ensuring a thorough mixing and an even distribution of the inoculum throughout the compost. The Phase 2 compost was spawned with a known Agaricus bisporus strain (such as Sylvan® A15 strain) before being filled into a Phase 3 tunnel and the colonisation of the compost was allowed to proceed for 15-16 days. Compost samples were taken at the end of Phase 1, Phase 2 and Phase 3 to assess the moisture content, pH and the rate of mycelial colonisation (laccase activity, this assay was performed only on Phase 3 compost).

Growing Trials to Confirm Effect of Compost Augmentation on Mushroom Yield and Quality

9 tonnes of the augmented compost and 9 tonnes of the control compost were each cased with mushroom casing on day 1. Initially, the CO₂ ranged between 8,000-12,000 ppm, temperature between 24-27° C. and high humidity (>90%) (stimulating the ideal conditions for vegetative growth). Once an optimal casing colonisation was obtained, conditions were changed to stimulate the development of the fruiting body. Specifically, the temperature was reduced to a range of 17−22° C. and the CO₂ to <2,000 ppm. A commercially available computer system (such as Fancom®) was used to regulate the CO₂, air temperature and humidity in the growth house. The compost temperature was monitored throughout the cropping cycle, to estimate the difference in the total microbial activity between the augmented and the control compost. The laccase activity of compost was monitored on a daily basis from day 12 of Phase 3 to the end of case run. 10 g of compost was mixed with distilled water and agitated for 20 minutes. The supernatant was collected and analysed as previously described. A higher laccase activity was correlated to a higher mushroom mycelium growth, and was generally in the range of 100-200 U/g_{DM} (units per gram of dry matter) at the end of Phase 3. The cropping cycle lasted 39 days and mushrooms from three flushes were assessed for yield (kg of mushrooms/m² of compost) and quality (dry matter content, piece weight and cup diameter).

### Example 1: Isolation, Identification and Characterisation of Bacillus licheniformis Strains MM76 and MM172

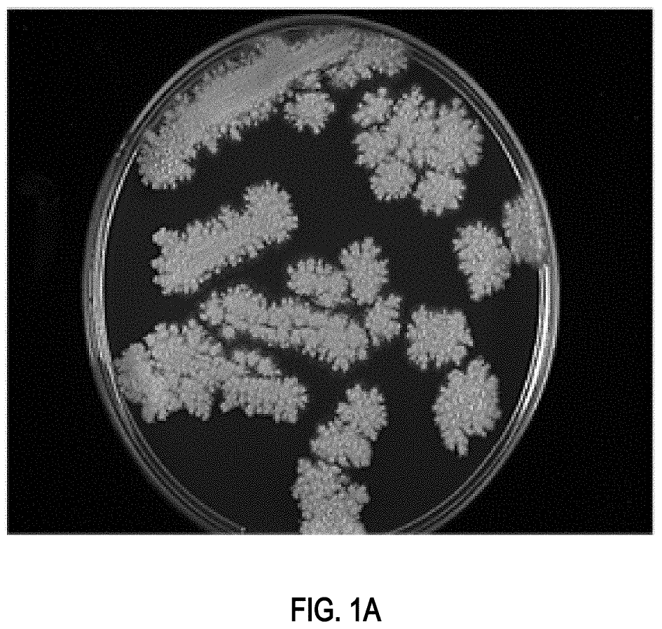

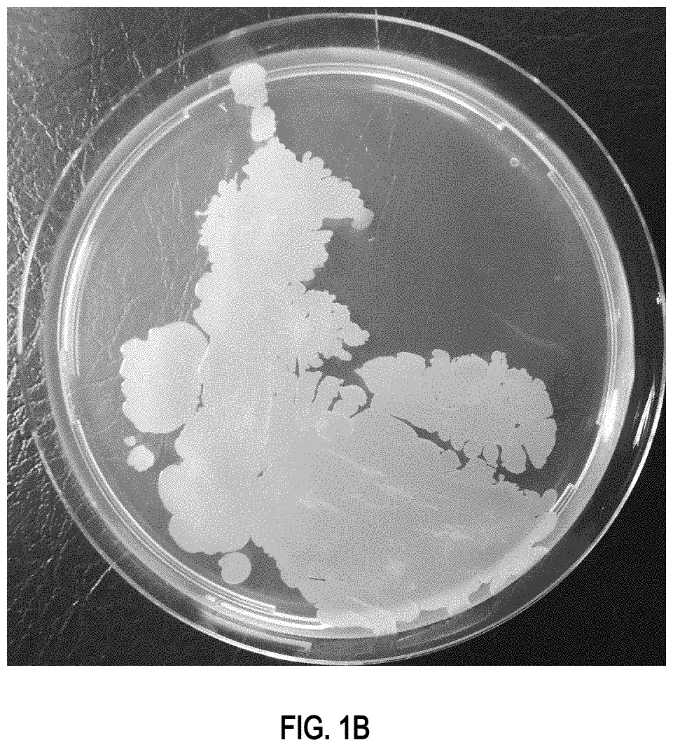

Strain MM76 was isolated using trypticase soy agar (TSA) media plates, incubated at 55° C. for 24 hrs, from Phase 1 mushroom compost (composed of fermented wheat straw, chicken litter) from Kabeyun Compost, Co. Monaghan, Ireland. Individual colonies were re-streaked onto TSA media plates to obtain pure bacterial cultures. The colony and cellular morphology of MM76 were assessed (FIGS. 1A and 1B) and MM76 strain was determined to be a gram positive, catalase positive and oxidase positive rod shaped bacillus, as summarised below in Table 1. Sequencing of the 16S ribosomal RNA indicated that MM76 was a Bacillus licheniformis strain. On Nov. 16, 2016, MM76 was deposited under the Budapest Treaty in NCIMB and was confirmed as a Bacillus licheniformis strain (Accession number: NCIMB 42690).

TABLE 1
COLONY MORPHOLOGY

| | |
|---|---|
| Colour | White |
| Surface | Rough |
| Form | Irregular |
| Elevation | Flat |
| Margin | Curled |

CELL MORPHOLOGY

| | |
|---|---|
| Cell shape | Rods |
| Other | Short bacilli, with filaments |

BIOCHEMICAL CHARACTERISATION

| | |
|---|---|
| Catalase | POS |
| Oxidase | POS |
| Gram Stain | POS |

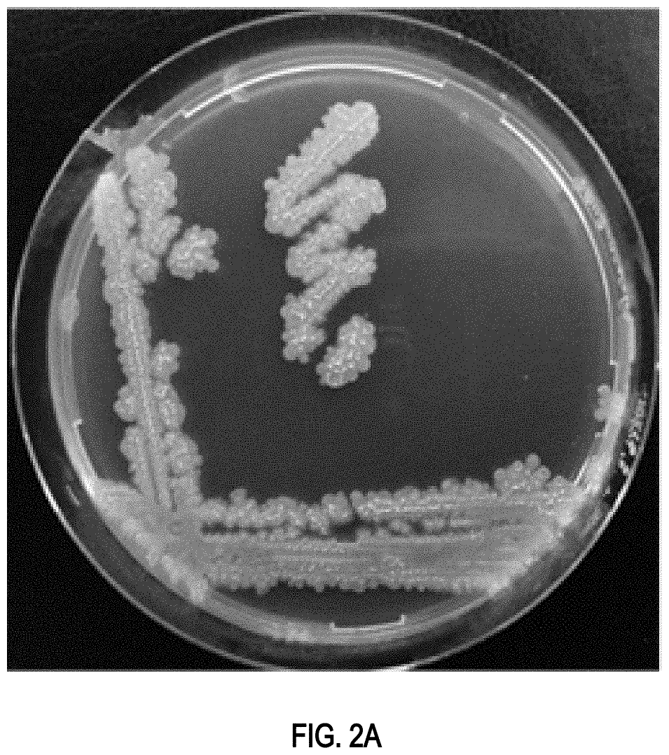

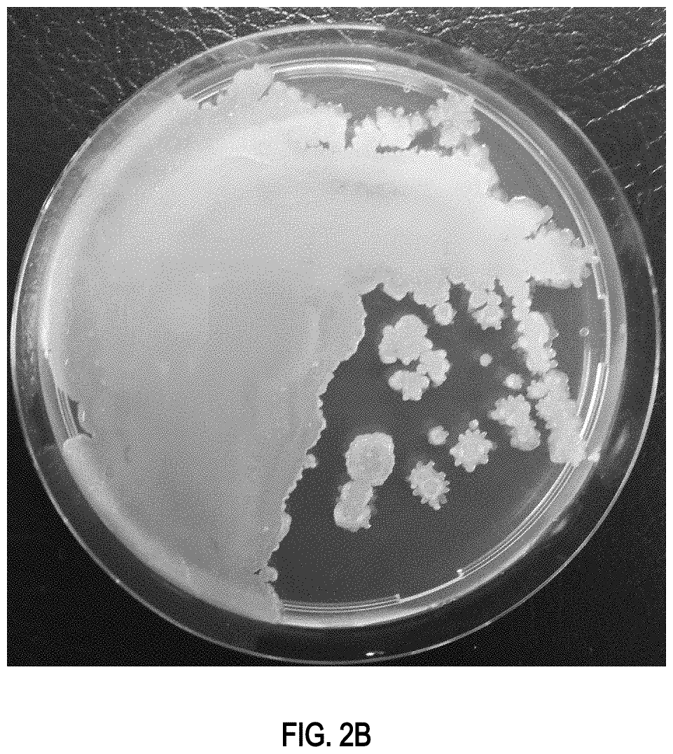

Strain MM172 was isolated using Actinomycete Isolation Agar (AA) media plates, incubated at 37° C. for 24 hrs, from Phase 1 compost (composed of fermented wheat straw and chicken litter) from a bunker at Carbury Compost, Co. Kildare, Ireland. Individual colonies were re-streaked onto TSA plates to obtain pure bacterial cultures. The colony and cellular morphology of MM172 were assessed (FIGS. 2A and 2B) and it was determined to be a gram positive, catalase positive and oxidase positive rod shaped bacillus, as summarised below in Table 2. Sequencing of the 16S ribosomal RNA indicated that MM172 was a Bacillus licheniformis strain. On the Nov. 16, 2016, MM172 was deposited under the Budapest Treaty in NCIMB and was confirmed as a Bacillus licheniformis strain (Accession number: NCIMB 42691).

TABLE 2
COLONY MORPHOLOGY

| | |
|---|---|
| Colour | White |
| Surface | Rough |
| Form | Filamentous |
| Elevation | Raised |
| Margin | Filamentous |

CELL MORPHOLOGY

| | |
|---|---|
| Cell shape | Rods |

BIOCHEMICAL CHARACTERISATION

| | |
|---|---|
| Catalase | POS |
| Oxidase | POS |
| Gram stain | POS |

### Example 2: In Vitro Assessment of the Ability of Bacillus licheniformis Strains MM76 and MM172 to Degrade Complex Carbohydrates in Mushroom Compost

MM76 was the best thermophilic candidate for a microbial composition, achieving the highest results in all of the functional tests, with the exception of the hemicellulose test, as shown in Table 3. The mesophilic MM172 also achieved high scores in the majority of the functional tests, with the exception of the hemicellulose degradation and ammonia assimilation tests, as shown in Table 3. The high scoring of MM76 and MM172 in the in vitro tests resulted in their inclusion in the microbial composition. An in vitro assay was conducted to test if the growth of either isolate inhibited the other, and confirmed that although there was limited (non-significant) inhibition in one of the 6 tested media, there was no inhibition in the majority of tested media environments. The results are shown in Table 4.

TABLE 3

| | Lignin | | Hemicellulose | Cellulose | Protein | Ammonia | | |
|---|---|---|---|---|---|---|---|---|
| Isolate | LB + CR | LB + TB | XY + CR | CMC + CR | SM | AA | Total | Species |
| MM76 | +++ | +++ | ++ | +++ | +++ | +++ | 17 | Bacillus |
| | | | | | | | | licheniformis |
| MM172 | +++ | +++ | ++ | +++ | +++ | ++ | 16 | Bacillus |
| | | | | | | | | licheniformis |

LB + CR: Luria Bertani Agar medium stained with Congo Red
LB + TB: Luria Bertani Agar medium stained with Toluidine Blue
XY + CR: Xylan Agar medium flooded with Congo Red
CMC + CR: Carboxymethylcellulose Agar medium flooded with Congo Red
SM: Skim Milk Agar
AA: Ammonia Agar

TABLE 4

| | | | Key: (0) no growth, (−) growth overlap, (+) Small growth, (++) medium growth | | | | | |
|---|---|---|---|---|---|---|---|---|
| | | | (+++) good growth | | | | | |
| Optimal | Optimal | | | | | | Skim | |
| temp. 55° C. | temp. 37° C. | Assay | TSA | TSA | CMC | CMC | Milk | Skim Milk |
| Isolate 1 | Isolate 2 | temp. (° C.) | Isolate 1 | Isolate 2 | Isolate 1 | Isolate 2 | Isolate 1 | Isolate 2 |
| MM76 | MM172 | 37 | +++ | +++ | +++ | +++ | +++ | +++ |
| MM76 | MM172 | 55 | +++ | +++ | +++ | +++ | + | +++ |

### Example 3: In Situ Assessment of the Ability of Bacillus licheniformis Strains MM172 and MM76 to Degrade Complex Carbohydrates in Mushroom Compost

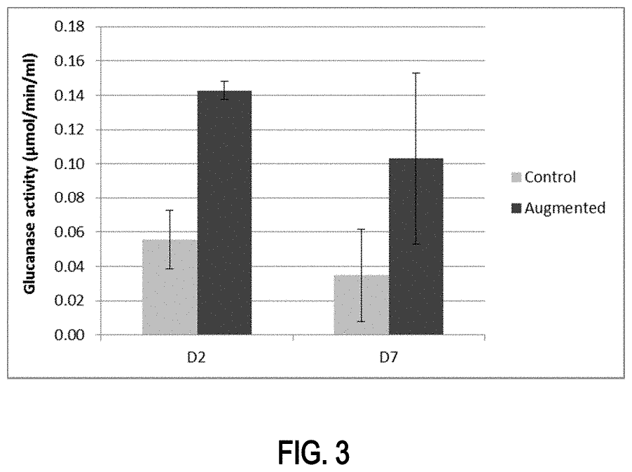

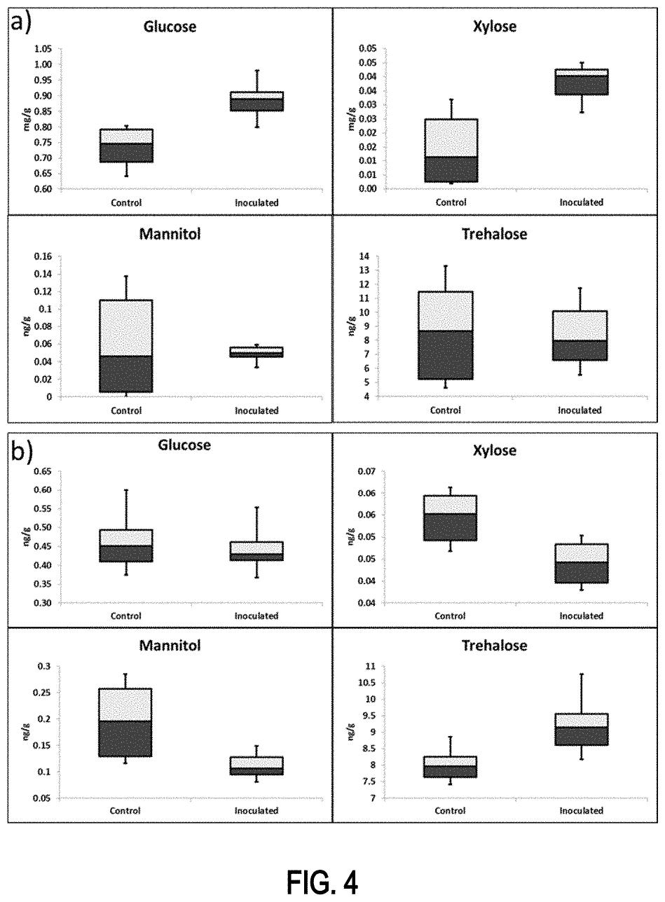

Control and augmented compost produced using the bench composting system was assessed for its level of complex carbohydrate degradation. The augmented compost comprised strains MM172 and/or MM76. Glucanase activity measured on day 2 and day 7 of the composting process confirmed that the augmented compost had higher levels of cellulose degrading activity than the control compost (FIG. 3). This resulted in higher levels of the breakdown products of cellulose, glucose and trehalose (FIG. 4).

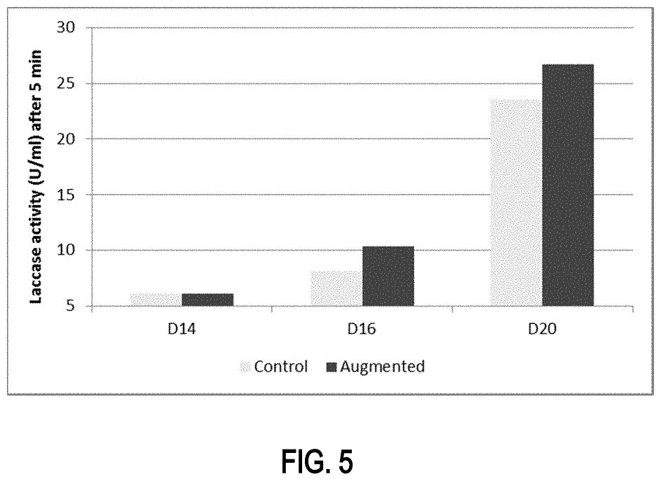

Mycelial colonisation assays were used to confirm that the compost produced using the BCS could be colonised by Agaricus bisporus over 21 days. Laccase is an enzyme that is produced by Agaricus bisporus in order to release simple carbohydrates. It has been reported to be a good indication of the success of the growth of the mushroom mycelium. Samples of compost were taken from replicate plates (containing 15 g of compost) for analysis of laccase activity (FIG. 5). At day 14, the laccase activity was similar in both control and augmented compost. However, on days 16-20, the laccase activity was consistently highest in the augmented compost. This suggested that the rate of mycelial colonisation was quicker in the augmented compost compared to the control composts.

### Example 4: Large Scale Trials to Confirm the Efficacy of Bacillus licheniformis Strains MM76 and MM172 to Degrade Complex Carbohydrates in Mushroom Compost

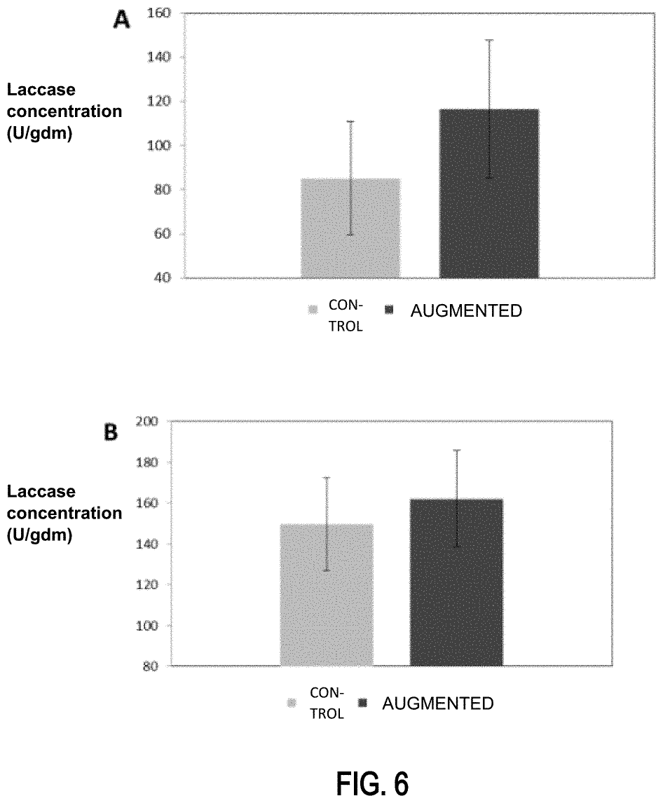

Quantification of the compost laccase activity was conducted from day 12 of Phase 3 composting to the end of case run, for a total of 8 days. Results confirmed that this enzyme activity was approximately 10% higher in the inoculated compost throughout this time period, indicating better mycelial colonisation of the compost (FIG. 6).

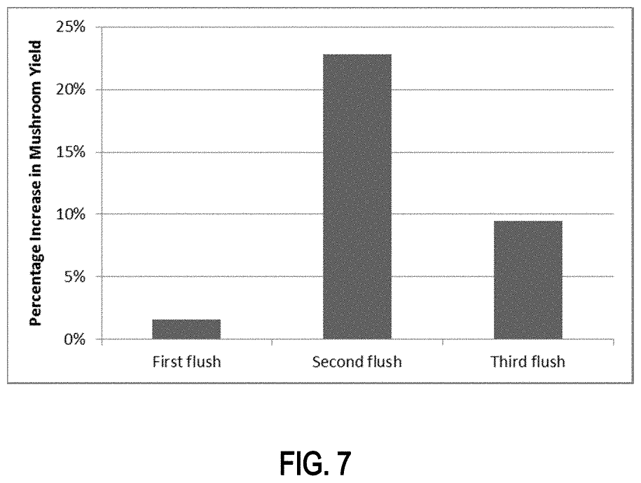

Mushrooms were harvested from each of three flushes and the yield was assessed (FIG. 7). The augmented compost yielded approximately 8% higher than the control compost, with the majority of the increase observed in second flush.

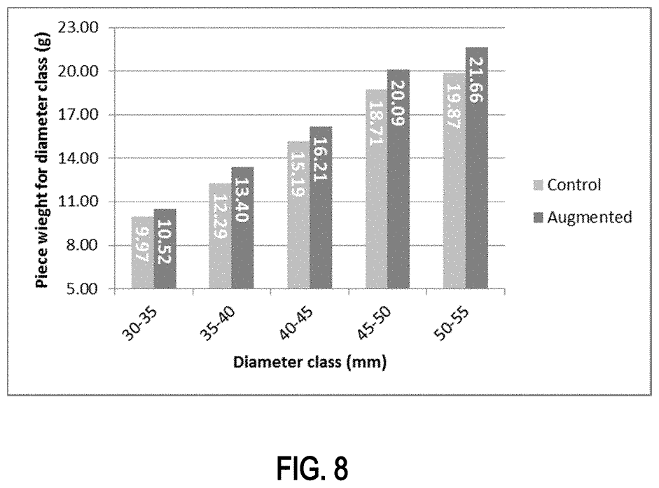

Piece weight and cup diameter of mushroom samples were measured (FIG. 8). Mushrooms belonging to the “closed cup” category (class diameter ranging between 30-55 mm) from augmented compost were heavier than control. The increase in piece weight of mushrooms cultivated on augmented compost was 5-9% compared to those cultivated on control compost.

### REFERENCES

Colak, M. 2004. Temperature profiles of Agaricus bisporus in composting stages and effects of different composts formulas and casing materials on yield. African Journal of Biotechnology, Vol. 3 (9), pp. 456-462.
Jiang, H., Dong, H., Zhang, G., Yu, B., Chapman, L. R. and Fields, M. W. 2006. Microbial diversity in water and sediment of Lake Chaka, an athalassohaline lake in northwestern China. Applied and Environmental Microbiology, Vol. 72 (6), pp. 3832-3845.
Johannes, C. and Majcherczyk, A. 2000. Laccase activity tests and laccase inhibitors. Journal of Biotechnology, Vol. 78 (2), pp. 193-199.
King, Brian C., Donnelly, Maria K., Bergstrom, Gary C., Walker, Larry P., Gibson, Donna M. 2009. An optimized microplate assay system for quantitative evaluation of plant cell wall-degrading enzyme activity of fungal culture extracts. Biotechnology and Bioengineering, Vol. 102, pp. 1033-1044.
Noble, R., Fermor, T. R., Evered, C. E. and Atkey, P. T. 1997. Bench-scale preparation of mushroom substrates in controlled environments. Compost Science and Utilization, Vol. 5 (3), pp. 32-43.

1. A bacterial composition comprising at least one strain of Bacillus licheniformis that is capable of (a) degrading complex carbohydrates, lignin, and protein; and (b) assimilating ammonia; wherein the at least one strain of Bacillus licheniformis comprises a gram positive, catalase positive and oxidase positive rod shaped strain that has a white coloured, rough surfaced colony morphology.

2. The bacterial composition according to claim 1, wherein the at least one strain of Bacillus licheniformis comprises one or more of:
a strain having a white colour, rough surface, irregular form, flat elevation, and curled margin colony morphology; and
a strain having a white colour, rough surface, filamentous form, raised elevation, and filamentous margin colony morphology.

3. The bacterial composition according to claim 1, wherein the at least one strain of Bacillus licheniformis comprises one or more of Bacillus licheniformis strain deposited at NCIMB Ltd. with accession number NCIMB 42690, the Bacillus licheniformis strain deposited at NCIMB Ltd. with accession number NCIMB 42691, and combinations thereof.

4. The bacterial composition according to claim 3 comprising the Bacillus licheniformis strain deposited at NCIMB Ltd. with accession number NCIMB 42690 and the Bacillus licheniformis strain deposited at NCIMB Ltd. with accession number NCIMB 42691.

5. A kit for preparing augmented compost and/or farming, the kit comprising the bacterial composition of claim 1.

6. The kit of claim 5, further comprising instructions for use thereof.

7. A method for preparing augmented compost, the method comprising the steps of:
providing the bacterial composition according to claim 1;
providing compost; and
inoculating the compost with the bacterial composition.

8. The method of claim 7, wherein the step of inoculating the compost with the bacterial composition is carried out under temperature-controlled conditions.

9. The method of claim 7, wherein the step of inoculating the compost with the bacterial composition is carried out under aerobic conditions.

10. Augmented compost comprising the bacterial composition of claim 1.

11. A method for providing augmented compost as a nutrient source for farming, the method comprising the steps of:
providing the augmented compost prepared according to claim 7;
providing one or more of fungus, mycelium, spawn, spore, plant, cutting, turf, and seed; and
cultivating the one or more of fungus, mycelium, spawn, spore, plant, cutting, turf, and seed in an environment comprising the augmented compost.

12. The method according to claim 11, comprising the steps of:
providing the augmented compost prepared according to claim 7;
providing at least one Agaricus bisporus mushroom, mycelium, spawn, and/or spore; and
cultivating the at least one Agaricus bisporus mushroom, mycelium, spawn, and/or spore, in an environment comprising the augmented compost.

13. Augmented compost comprising the bacterial composition of claim 3.

14. Augmented compost comprising the bacterial composition of claim 4.

15. A method for preparing augmented compost, the method comprising the steps of:
providing the bacterial composition according to claim 3;
providing compost; and
inoculating the compost with the bacterial composition.

16. A method for preparing augmented compost, the method comprising the steps of:
providing the bacterial composition according to claim 4;
providing compost; and
inoculating the compost with the bacterial composition.

17. A method for providing augmented compost as a nutrient source for farming, comprising the steps of:
providing the augmented compost prepared according to claim 15;
providing one or more of fungus, mycelium, spawn, spore, plant, cutting, turf, and seed; and
cultivating the one or more of fungus, mycelium, spawn, spore, plant, cutting, turf, and seed in an environment comprising the augmented compost.

18. A method for providing augmented compost as a nutrient source for farming, comprising the steps of:
providing the augmented compost prepared according to claim 16;
providing one or more of fungus, mycelium, spawn, spore, plant, cutting, turf, and seed; and
cultivating the one or more of fungus, mycelium, spawn, spore, plant, cutting, turf, and seed in an environment comprising the augmented compost.


SEQUENCE LISTING (4 sequences)

SEQ ID NO: 1 - DNA, 21 bp, Artificial Sequence
  Synthetic oligo
agagtttggatcmtggctcag

SEQ ID NO: 2 - DNA, 20 bp, Artificial Sequence
  Synthetic oligo
cggttaccttgttacgactt

SEQ ID NO: 3 - DNA, 617 bp, Artificial Sequence
  Bacillus licheniformis MM76 16S partial sequence
cttgctcccttaggtcagcggcggacgggtgagtaacacgtgggtaacctgcctgtaagactgggataactccgggaaaccggggctaataccggatgcttgattgaaccgcatggttcaatcataaaaggtggcttttagctaccacttacagatggacccgcggcgcattagctagttggtgaggtaacggctcaccaaggcgacgatgcgtagccgacctgagagggtgatcggccacactgggactgaaacacggcccacactcctacgggaggcagcagtaaggaatcttccgcaatggacgaaagtctgacggaacaacgccgcgtgagtgatgaaggttttcggatcgtaaaactctgttgttagggaagaacaagtaccgttcgaatagggcggcaccttgacggtacctaaccagaaagccacggctaactacgtgccagcggccgcggtaatacgtaagtggcaagcgttgtccggaattattgggcgtaaagcgcgcgcagcggtttcttaagtctgatgtgaaagcccccggctcaaccggggagggtcattggaaactggggaacttgagtgcagaagaagaaaatggaattccacgtgtacgg

SEQ ID NO: 4 - DNA, 843 bp, Artificial Sequence
  Bacillus licheniformis MM172 16S partial sequence
cttgctcccttaggtcagcggcggacgggtgagtaacacgtgggtaacctgcctgtaagactgggataactccgggaaaccggggctaataccggatgcttgattgaaccgcatggttcaatcataaaaggtggcttttagctaccacttacagatggacccgcggcgcattagctagttggtgaggtaacggctcacagaggcgacgatgcgtagccgacctgagagggtgatcggccacactgggactgagacacggcccagactcctacgggaggcaccagtagggaatcttccgcaatggacgaaagtctgacggagcaacgccgcgtgagtgatgaaggttttcggatcgtaaaactctgttgttagggaagaacaagtaccgttcgaatagggcggcaccttgacggtacctaaccagaaagccacggctaactacgtgccagcagccgcggtaatacgtaggtggcaagcgttgtccggaattattgggcgtaaagcgcgcgcaggcggtttcttaagtctgatgtgaaagcccccggctcaaccggggagggtcattggaaactggggaacttgagtgcagaagaggagagtggaattccacgtgtatcggtgaaatgcgtagagatgtggaggaacaccagtggcgaaggcgactctctggtctgtaactgacactgaggcgcgaaagcgtgggagcgaacaggattagataccctggtagtccacgccgtaacgatgagtgctaagtgttagaggtttccgccctttatgctgcagcaacgcattaagcactccgcctggggagtacggtcgcaagactgaactcaaggaatt